(19) United States
(12) Patent Application Publication
(10) Pub. No.: US 2007/0078433 A1
(43) Pub. Date: Apr. 5, 2007

(54) Process and arrangement for an application catheter

(75) Inventors: Michael Schwager, Winterthur (CH); Karl Konstantin Haase, Speyer (DE); Christian Herdeg, Tubingen (DE)

Correspondence Address: BUCHANAN, INGERSOLL & ROONEY PC, POST OFFICE BOX 1404, ALEXANDRIA, VA, 22313-1404, US

(73) Assignee: ACROSTAK CORP., CH-Winterthur (CH)

(21) Appl. No.: 11/409,120

(22) Filed: Apr. 24, 2006

Related U.S. Application Data
(63) Continuation of application No. PCT/EP04/11866, filed on Oct. 20, 2004.

(30) Foreign Application Priority Data
Oct. 23, 2003 (EP) 03024341.4

(51) Int. Cl. A61M 31/00

(52) U.S. Cl. 604/500

(57) ABSTRACT

The invention relates to a process for administering a medication in a blood vessel using a balloon catheter with a balloon which has at least one flank. An inflow direction of the medication is established (i.e., takes place) at an angle α from 0° to 89°, the angle α being formed from the flow direction of the blood in the blood vessel and the flank of the balloon.

### RELATED APPLICATION

This application claims priority under 35 USC §119 to European Application No. 03024341.1, filed Oct. 23, 2003, and under 35 USC §120 to International Application No. PCT/EP2004/001866, the contents of which are incorporated herein by reference.

### BACKGROUND

A process and an arrangement are disclosed for an application catheter for administration of a medication in a blood vessel by means of a balloon catheter with a balloon which has at least one flank.

Processes and arrangements of application catheters are used to administer a medication at a desired location, with the objective of achieving an optimum action. This idea of local application underlies the idea of achieving better results with a high local concentration of the active ingredient and fewer systemic side effects.

The use of a medication-coated stent is known. Balloon systems are also known.

Publication 2003/0045860 A1 discloses such a process and arrangement of this type. It is two sequentially arranged, expandable balloons, with outside walls which press against the inside wall of the vessel to be treated. The point to be treated on the blood vessel is located between the two balloons. The area with a much smaller diameter located in the intermediate area of the two balloons is perforated. The medication is introduced in the conventional manner through these perforations, in doing so the flow direction of the medication runs directly, i.e. at an angle α of 90°, to the flow direction of the blood. The medication thus travels with maximum pressure to the blood vessel location to be treated.

A different process and a different arrangement of an application catheter are described in publication U.S. Pat. No. 5,611,775. Here it is a device having of an expandable balloon, the balloon being perforated on the contact surface of the balloon with the inside wall of the vessel for feed of the medication.

These processes can have limits with respect to the efficiency of the catheter-supported local therapy by a generally low transfer of the active ingredient and rapid washing of the substance out of the vessel wall. Since the physical possibilities of introducing an active ingredient by means of the catheter into the vessel wall can be limited and fluctuate between the two possibilities of passive application (vessel wall is bathed in the active ingredient) on the one hand and active application (active ingredient is introduced with maximum possible pressure into the vessel wall), a suitable process and suitable arrangement were sought which make it possible to move continuously between the two active and passive possibilities. Otherwise in these known catheter arrangements complete emptying of the treatment space is not guaranteed, with the consequence that the medication is partially rinsed away by the blood and must be broken down by the body. This can cause unwanted systemic dispersions of the medication in the body.

### SUMMARY

A process and an arrangement are disclosed for an application catheter for administering a medication in a blood vessel by means of a balloon catheter in which longer application times are possible while avoiding vascular trauma. Furthermore, complete emptying of the application space can be achieved. An exemplary approach is specifically suited for a balloon-supported application according to the pharmacological profile of a medication.

The inflow direction of the medication can be at an angle α from 0 ° to 89°, the angle being formed from the flow direction of the blood into the blood vessel and the flank of at least one balloon. Furthermore, the balloon can have an additional lumen with an open end located between the distal end of the balloon and the proximal end of the adjacent balloon.

Exemplary embodiments can deliver an active ingredient in a high concentration in a uniform manner which does not traumatize the vessel over a defined interval on site. For example, this fact can be decisive for delivery of Paclitaxel. Paclitaxel leaves behind its cellular action, specifically a change of the cytoskeleton, even if the substance itself has been long washed out. Harmful side effects can be avoided, at least reduced. In this connection, for example, the following additional substances suitable for this process are named: Sirolimus, Everolimus, and stem cells.

The distal and proximal balloon can be unfolded and inflated with the medication at the same time in the following manner:

The medication is injected into the catheter through a shaft. In this way the balloon unfolds first into its shape and by its dumbbell configuration (distal and proximal balloon) develops a sealing action on the two sides of the area to be treated. That the balloon first unfolds is technically achieved solely by the total cross section of the perforations being relatively small. Thus the medication emerging from the perforations without unfolding of the balloon will be prevented. The application space is emptied by using negative pressure. The medication is drawn back again through the perforations. In doing so the volume of the application space must be relatively small compared to the total volume of the catheter.

The balloon flanks pointed toward the diseased vessel areas can be provided with perforations, the steepness of the flanks being adjustable.

The angle α can be adjusted depending on the application and/or medication. Thus the steepness of the perforated flanks of a balloon determines the pressure with which the medication is applied to the area to be treated. In this way it becomes possible to set the penetration depth and action interval of a medication. With this possibility of adjusting the angle the catheter arrangement can be aligned specifically to the substance to be administered and the special application.

According to an exemplary version the catheter arrangement has an additional lumen, with an open end located between the distal end of the balloon and the proximal end of the adjacent balloon. This can almost nearly prevent the blood flow in the blood vessel from being influenced. Continuity of the blood flow is ensured by suitable means.

### BRIEF DESCRIPTION OF THE DRAWINGS

The invention is described below by an exemplary embodiment.

FIG. 1 shows a schematic cross section of an exemplary catheter arrangement;

FIG. 2 shows the detail B from FIG. 1 enlarged;

FIG. 3 shows a cross section along section C-C from FIG. 1;

FIG. 4 shows a schematic cross section of an exemplary catheter arrangement with an additional lumen; and

FIG. 5 shows a cross section along the section A-A from FIG. 4.

### DETAILED DESCRIPTION

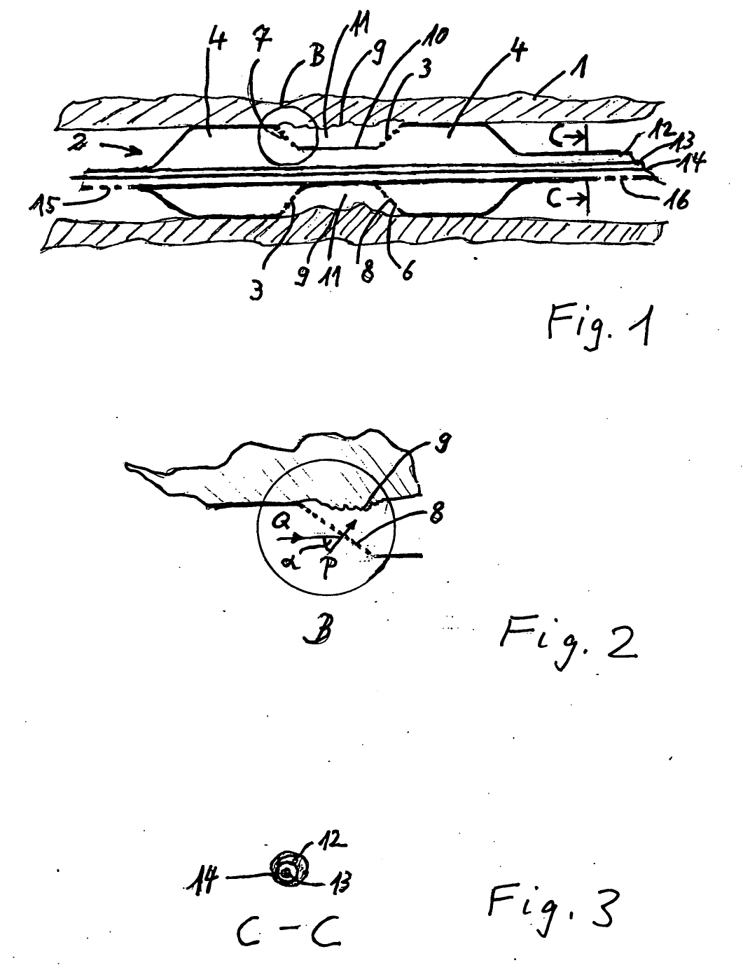

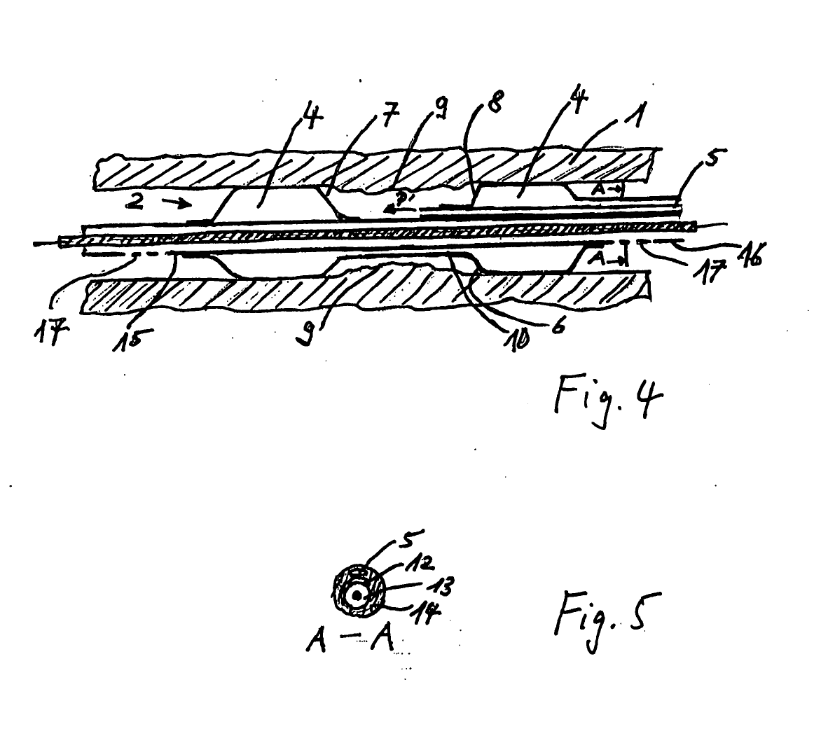

Reference number 1 relates to the blood vessel 1 which has an area 9 to be treated. In the blood vessel 1 a balloon catheter 2 comprising (e.g., consisting of) two balloons 4 is placed such that the area 9 to be treated is located between the two balloons 4. The cross section of the balloon 4 is larger than the cross section 10 of the area 11 between the balloons 4. The connection between the balloon 4 and the intermediate area 11 is made by means of flanks 3, both on the proximal end 7 and also on the distal end 6 of the balloons 4. The walls of the flanks 3 facing the areas to be treated 9 can be perforated for feed of medications, and intentionally not the wall of the intermediate area 11. FIG. 2 shows the detail B from FIG. 1. The angle α is defined as the angle established between the direction Q of the blood flow in the vessel 1 and the direction P of medication flow. The medication is fed through the perforations 8 in the flanks 3. Based on the angle α which is adjustable in the production of the balloon catheter, the impact pressure and thus the intensity of action (length and depth of action) can be specifically set depending on the application and medication. FIG. 3 shows the cross section of section C-C from FIG. 1. The inflation lumen 12, the wire lumen 13 and the guide wire 14 are apparent.

In FIG. 4 the catheter arrangement has an additional lumen 5 for separate medication delivery. The medication is introduced through the additional lumen 5 into the intermediate space 11 parallel to the inflow direction P of blood flow in direction P′. A curved end of the additional lumen 5 pointing into the treatment space is also conceivable. The medication is delivered at the desired angle and the associated impact pressure to the site to be treated.

The sectional drawing in FIG. 5 according to the section A-A from FIG. 4 shows the additional lumen 5, the inflation lumen 12, the wire lumen 13 and the lumen for the guide wire 14. There are perforations 17 both on the distal end 16 and also the proximal end 15 of the catheter arrangement. These perforations 17 are used to maintain and thus to equalize the blood flow in the blood vessel 1.

The process can also be used to treat other organ systems, such as for example the trachea, bronchial tubes, esophagus, efferent urinary tracts or gall ducts channels by means of specific medications.

Exemplary advantages include a process and a catheter arrangement for specific treatment relative to the application and medication properties.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

### REFERENCE NUMBERS

1 blood vessel
2 balloon catheter
3 flank
4 balloon
5 lumen
6 distal end
7 proximal end
8 perforations
9 treating region
10 cross section of intermediate space 11
11 intermediate space
12 inflation lumen
13 wire lumen
14 wire
15 proximal end
16 distal end
17 perforations
P inflow direction
Q flow direction of the blood in the vessel
α angle

1. Process for administering a medication in a blood vessel using a balloon catheter with a balloon which has at least one flank, comprising:
establishing an inflow direction P of medication at an angle α from 0 ° to 89°, the angle α being formed from a flow direction Q of blood in the blood vessel and the flank of the balloon.

2. Process for administration of a medication as claimed in claim 1, wherein the angle α can be adjusted depending on an application and/or type of medication.

3. Process as claimed in claim 2, wherein the catheter arrangement is configured specifically for a substance to be administered.

4. Process as claimed in claim 1, wherein an overall cross section of perforations are made such that the balloons 4 can be unfolded and inflated in a dumbbell shape.

5. Catheter arrangement for administering a medication in a blood vessel comprising:
a balloon catheter with a balloon which has at least one flank; and
an additional lumen with an open end located between a distal end of the balloon and a proximal end of the adjacent balloon, and which points into the treatment space.

6. Catheter arrangement as claimed in claim 5, comprising:
perforations located both on the proximal end and on the distal end of the catheter arrangement.

7. Catheter arrangement as claimed in claim 5, wherein the least one flank of the balloon has perforations for administering a medication.

8. Catheter arrangement as claimed in claim 5, wherein the flank and the flow direction Q of the blood form an angle α, the angle being 0° to 89°.
